(19) United States
(12) Patent Application Publication
(10) Pub. No.: US 2009/0280093 A1
(43) Pub. Date: Nov. 12, 2009

(54) COMPOSITIONS AND POPULATIONS OF CELLS OBTAINED FROM THE UMBILICAL CORD AND METHODS OF PRODUCING THE SAME

(75) Inventor: Hyman Friedlander, Kiryat Ono (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: The Regenerative Medicine Institute, Las Vegas, NV (US)

(21) Appl. No.: 12/280,566

(22) PCT Filed: Feb. 27, 2007
(86) PCT No.: PCT/IL2007/000259

§ 371 Date: Aug. 25, 2008

Related U.S. Application Data
(60) Provisional application No. 60/777,517, filed on Mar. 1, 2006.

(51) Int. Cl. A61K 35/12; C12N 5/06

(52) U.S. Cl. 424/93.7; 435/379; 435/374; 435/325

(57) ABSTRACT

The present invention relates to populations and compositions of stem and progenitor cells derived from the umbilical cord, and methods of obtaining the same. In some embodiments, one or more entire umbilical cords or sections thereof are subjected to a process where a cell population is derived without prior removal of any blood vessel. The population may be derived using mechanical and chemical means. The presently disclosed process may be applied to a single umbilical cord or to a plurality of umbilical cords, for example, as a batch process. Optionally, this process includes removing some or all cord blood before deriving the population. In some embodiments, presently disclosed cell populations include mesenchymal stem cells derived from Wharton's jelly and endothelial progenitor cells derived from a wall of a blood vessel of an umbilical cord. Optionally, the cell population includes stem cells derived from cord blood. The presently disclosed cell populations and compositions may be banked and/or used in a number of clinical or other applications. Exemplary applications include but are not limited to applications related to regenerative medicine, for screening compounds, for research, and for gene therapy.

### FIELD OF THE INVENTION

The present invention relates to populations and compositions of stem and progenitor cells derived from the umbilical cord, and methods of obtaining the same.

### BACKGROUND OF THE INVENTION

Cell therapy, the use of living cells as therapeutic agents, is an alternative approach to medicine and is presently being used for several clinical indications such as treatment of injured joints, chronic ulcers, corneal damage, large burns, neural damage and others. A unique population of cells, stem cells (SC), are of special interest due to their self-renewal capacity and their potential to differentiate and develop into several different cell lineages.

There are two major types of stem cells. Embryonic stem cells (ESCs) are derived from blastocycts which arise in a very early stage of embryonic development. ES cells can be grown in culture to large numbers but are difficult to control in their development and are accompanied by unresolved ethical problems. The second type of stem cell is the adult stem cell (ASC), which is found in various tissues of the adult body. Each tissue and organ in the body originates from a small population of ASCs which is committed to differentiate into the various cell types that compose the tissue. ASCs are a likely source of continuous normal tissue replenishment as well as recovery in case of damage or disease, throughout the life of the organism.

The first and most widely studied tissue in animals is the blood. Most if not all blood cells, including red blood cells, lymphocytes, monocytes, polymorphs, and platelets originate from hematopoietic stem cells (HSC) which are located in the bone marrow but are also found in the circulation and other organs.

HSC from either bone marrow, peripheral blood or cord blood, are widely used for replacement of ablated bone marrow, for treatment of malignant and genetic diseases. In addition to HSC, it was recently found that the bone marrow contains primitive stem cells that can differentiate into other tissues and organs. Some of the ASC in the bone marrow are part of the well characterized population termed mesenchymal stem cells that can differentiate into bone, cartilage and heart muscle cells but other pluripotent stem cells were also detected. Such ASC were isolated recently from cord blood, Wharton's Jelly matrix, adult peripheral blood, fat tissue and other organs and under various conditions can give rise to additional tissues such as blood vessels, bone, cartilage, muscle, liver, nerve cells as well as insulin secreting Langerhans cells.

Additional types of ASC were also identified in various tissues. Actually, every tissue and organ in the body probably contains stem cells that participate in intrinsic regeneration and repair during growth, trauma and disease.

Mesenchymal stem cells were described in adult human bone marrow. Human bone marrow was reported to be a source of pluripotent stem cells, in addition to the hematopoietic stem cells. Bone marrow derived hematopoietic stem cells were also reported to maintain pluripotent potential for non-hematopoietic tissues. Hematopoietic stem cells with pluripotent potential were also found in other tissues such as cord blood.

Stem cells from these various sources are being tested clinically for treatment of diseases such as ischemic heart diseases, neural injuries, neuro-degenerative diseases, diabetes, as well as other diseases that do not currently have effective treatments. Many additional disease indications are under investigation at their pre-clinical research stage. Currently however, major limitations in the use of adult stem cells include their scarce availability in adults and immunological barriers between individuals that may restrict their transplantation. To improve availability, several approaches have recently been developed that can be used to generate stem cells from bone marrow and cord blood in sufficient numbers for therapeutic use. Several types of pluripotent stem and progenitor cells have also been identified recently in normal adult peripheral blood. Methods for isolating these cells are based on their membrane markers and plastic adherence properties. Methods are also described for their ex vivo expansion. However, it remains unclear which cell population is responsible for each in vivo function, and in several cases, therapeutic activity of defined stem cell populations was not demonstrated and the origin of the therapeutic cells is still controversial.

Recently, a novel source of mesenchymal stem cells was reported to occur in the Whartons jelly, which is the matrix surrounding the vein and arteries of the umbilical cord. In addition, endothelial progenitor cells were found in the walls of the blood vessels as well as in the peri-vascular tissues. All these tissues may also contain various types of progenitor cells, fibroblasts, hematopoietic stem cells, endothelial progenitor cells as well as yet unidentified stem and progenitor cells.

In order to obtain these mesenchymal stem cells from an umbilical cord, the Wharton's jelly is first mechanically isolated from the umbilical cord, by draining umbilical cord blood and subsequently dissecting away the blood vessels of the umbilical cord (two veins and an artery). It is noted that Wharton's jelly-derived stem cells have been isolated and studied (see US 2004/0136967 of Weiss et al.), and have been identified as useful for transplant, as a vehicle of introducing genes and gene products in vivo, and for various research and/or screening applications. Despite these promising findings, to date, Wharton's jelly-derived stem cells have, unfortunately, not been extracted and banked on a large scale, and it does not appear that this situation will change in the near future. This is, in part, due to the number of man-hours that must be invested to manually remove the umbilical cord blood vessels from each umbilical cord.

US 2005/0148074 of Davies et al discloses a Wharton's jelly extract comprising human progenitor cells that is obtained by enzymatic digestion of the perivascular tissue proximal to the vasculature of the human umbilical chord. It is disclosed that the extract is essentially free from cells of umbilical cord blood, epithelial cells or endothelial cells of the UC and cells derived from the vascular structure of the cord, where vascular structure is defined as the tunicae intima, media and adventia of arteriolar or venous vessels.

Sarugaser et al (Stem Cells 2005; 23:220-229) discloses that Human Umbilical Cord Perivascular (HUCPV) Cells is a source of mesenchymal progenitors cells that can potentially generate multiple therapeutic doses of cells for cell-based therapies, and thus represent a significant alternative to BM in the treatment of pathologies associated with the connective tissues of the human body.

Thus, there is an ongoing need for methods of obtaining stem cells and/or progenitor cells from the umbilical cord which may be readily applied in a clinical setting, on a large scale. Furthermore, there is an ongoing need for compositions of umbilical cord-derived cells for use in regenerative medicine and other applications.

### SUMMARY OF THE INVENTION

The present inventors are disclosing for the first time that, surprisingly, it is possible to obtain a useful composition and/or population of stem cells including Wharton's jelly-derived mesenchymal cells by extraction (for example, mechanical and/or enzymatic extraction) from umbilical cords (i.e. an entire umbilical cord or a section thereof) without prior removal of one or more of the blood vessels (for example, without prior removal of one or more veins) that are a part of the umbilical cord. In exemplary embodiments, the presently disclosed composition and/or cell population includes cells derived from umbilical cord matrix, umbilical cord perivascular tissue, and umbilical cord veins. Optionally, the presently disclosed composition and/or cell population includes cells derived from umbilical cord blood

In some embodiments, for a given umbilical cord, the method is carried out without prior removal of any blood vessel (or without prior removal or any vein).

Not wishing to be bound by theory, it is noted that the presently disclosed methods for obtaining compositions of cells including Wharton's jelly-derived mesenchymal cells and other stem cells and/or progenitor cells may be easier to carry out than methods which require prior removal of the blood vessels. Thus, the presently disclosed methods and compositions obviate the need for extensive mechanical processing of umbilical cord tissue when extracting stem cells, and may facilitate the harvesting of different types of stem cells from umbilical cords on a larger scale.

Not wishing to be bound by theory, it is now disclosed that the presently disclosed methods, with no attempt or minimal attempts to isolate or enrich for defined cell populations, may provide a method that is easier, faster, and less costly than previously-disclosed methods, which place an emphasis on cell purification and/or pre-removing the umbilical cord blood vessels from the umbilical cord matrix.

In some embodiments, the presently disclosed method may be carried out on a plurality of umbilical cords (or sections from a plurality of umbilical cords) as a batch process (for example, by first providing the plurality of umbilical cords within a vessel or container, and then mechanical disrupting (for example, substantially simultaneously) the umbilical cords within the vessel container). In some embodiments, the batch is carried out on a large number of umbilical cords (for example, substantially simultaneously), for example, at least 5 umbilical cords, at least 10 umbilical cords, etc.

Typically, the resultant compositions and cell populations obtained by the at least some of the presently-disclosed methods includes a mixture of mesenchymal stem cells (for example, Wharton's jelly-derived mesenchymal stem cells), endothelial progenitor cells (for example, derived from umbilical cord blood vessels) and optionally, hematopoietic stem cells (HSC) (for example, from umbilical cord blood).

Furthermore, the present inventor is disclosing, for the first time, novel compositions of cells including mixtures of mesenchymal stem cells, endothelial progenitor cells, and optionally hematopoietic stem cells. In some embodiments, a ratio between a number of mesenchymal stem cells and endothelial progenitor cells in the cell composition and/or cell population is substantially equal to (for example, within a 30% tolerance, or within a 20% tolerance, or within a 10% tolerance, or within a 5% tolerance) the naturally occurring ratio within umbilical cords. In different embodiments, this ‘natural occurring ratio’ may be measured for the case where the umbilical cord contains substantially all umbilical cord blood or substantially no umbilical cord blood

It is further disclosed that certain presently disclosed compositions and cell populations may have improved therapeutic effectiveness (i.e. due to a biological synergy) in the treatment of certain diseases and tissue regeneration treatments over their more purified counterpart cell populations.

In some embodiments, the population of cells including tissue-derived cells are provided ‘as is’ with no or very limited attempts at purification of the population of cells. It is now disclosed for the first time a method of deriving a population of cells comprising the steps of (a) obtaining one or more umbilical cords (i.e. one or more entire umbilical cords or a sections thereof), each umbilical cord comprising at least one respective umbilical cord blood vessel and respective umbilical cord matrix, (b) for each umbilical cord, mechanically disrupting at least a portion of at least one respective umbilical cord blood vessel and at least a portion of respective umbilical cord matrix to produce a mixture including umbilical cord blood vessel matter (i.e. matter derived from umbilical cord blood vessels) and umbilical cord matrix matter; and (c) deriving (for example, by extracting and collecting) the population of cells including umbilical cord matrix-derived cells and umbilical cord blood vessel-derived cells from said mixture.

In some embodiments, the method is carried out, for each umbilical cord, without removal of at least one respective blood vessel (or alternatively, for each umbilical cord, without removal of any respective blood vessels).

It is noted that in different embodiments, “mechanical disrupting” of the umbilical cord may include at least one of mincing, grinding, crushing, cutting, mashing, chopping, and squeezing. In different embodiments, the mechanical disruption may be carried out using at least one of a surgical knife, medimachine, scissors, or other device.

In some embodiments, the method (e.g. the mechanical disrupting) includes chopping said umbilical cord into a plurality of small pieces.

There is no explicit limitation on the size of the “small pieces.” In some cases, small pieces of different sizes may be produced. Typically, the “small pieces” include pieces whose volume is less than one half of the volume. of the respective component (i.e. pieces of blood vessel whose volume is less than one half of the volume of a blood vessel, pieces of Wharton's Jelly whose volume is less than one half of the volume of Wharton's jelly of an umbilical cord), while still being visible to the naked eye (i.e. having a characteristic dimensions that is at least 0.5 mm).

In exemplary embodiments, the small pieces may includes pieces of all difference sizes, including but not limited to small pieces whose characteristic dimensions are between 0.5 mm and 2 cm.

In some embodiments, the method (e.g. the mechanical disrupting) includes forming a paste-like material from the umbilical cord and deriving said population from said paste-like material.

In some embodiments, the presently disclosed “mixture” may include a plurality of pieces of umbilical cord blood vessel matter (i.e. pieces of the umbilical cord blood vessel) and a plurality of pieces of umbilical cord matrix matter.

In some embodiments, the presently-disclosed “mixture” may include pieces of perivascular tissue.

In some embodiments, a presently disclosed cell population may include cells derived from the perivascular tissue (for example, mesenchymal cells and/or progenitor cells)

In some embodiments, the mechanical disrupting essentially destroys the original form of the cord vessel and/or the umbilical cord matrix.

Optionally, the presently-disclosed mixture is at least partially homogenized.

In some embodiments, the umbilical cord (or section thereof) from which a majority or any portion or substantially all cord blood is removed before the mechanical disrupting, or before the deriving.

Alternatively, the cord blood is either left within the cord blood vessels (i.e. the cord blood veins), or after removal from the cord blood vessels, is re-introduced into the cord blood vessels and/or into the presently-disclosed mixture and/or into a composition or mixture including the presently-disclosed derived population of cells.

It is now disclosed for the first time a method of deriving a population of cells comprising (a) obtaining one or more umbilical cords, each umbilical cord comprising at least one respective umbilical cord blood vessel and a respective umbilical cord matrix, and (b) deriving (for example, by extracting and collecting) the population of cells from said umbilical cord without prior removal of at least one respective blood vessel and/or without prior removal at any respective blood vessel and/or without prior removal of at least one respective vein and/or without prior removal of any respective vein.

Thus, in some embodiments, the mixture from which the population of cells (for example, using any of the presently disclosed methods) is derived includes umbilical cord blood. In some embodiments, the mixture includes a majority of umbilical cord blood of said umbilical cord.

According to some embodiments, the derived population of cells (for example, using any of the presently disclosed methods) includes at least mesenchymal cells (for example, Wharton's jelly-derived mesenchymal cells) and endothelial progenitor cells.

Optionally, the derived population (for example, using any of the presently disclosed methods) of cells further includes hematopoietic stem cells (for example, derived from cord blood).

According to some embodiments, the presently disclosed population of cells (for example, using any of the presently disclosed methods) includes progenitor cells derived from perivascular tissue of the umbilical cord (for example, immuno-incompoetenct progenitor cells, for example, progenitor cells capable of giving rise to one of bone cells and fat cells).

In some embodiments, the deriving for (using any of the presently disclosed methods) includes extracting by chemical means (for example, by contacting with a chemical agent) such as enzymatic extraction (for example, using collagenase, trypsin, elastase, or any other enzyme as well as chemical agents such as EDTA).

In some embodiments, any of the presently-disclosed methods further includes the step of administering a therapeutic compound (i.e. a mixture including the presently disclosed population of cells and a pharmaceutically acceptable carrier, for example, a carrier for targeted delivery to a particular site, for example to a tissue site) comprising the population of including said population of cells to a patient in need thereof. According to particular embodiments, the cells of the derived population are not activated ex vivo and/or not purified before administration.

In some embodiments, “purification” is defined to include processes whereby cells that are not stem cells and/or progenitor cells (i.e. mature cells) are removed from a mixture and/or population and/or composition of cells.

According to some embodiments, the cells (for example, derived, using any of the presently disclosed methods) are associated with a biocompatible carrier before being administered and/or transplanted to the patient in need thereof. In some embodiments, the cells are associated with a medical implant that is implanted into the patient.

In some embodiments, the method further includes cryopreserving at least a portion of said population of cells. According to particular embodiments, the cells of the derived population are not activated ex vivo and/or not purified (or purified only to a limited extent) before cryopreserving.

According to some embodiments, the presently disclosed method includes (e) thawing the cryopreserved cells, and (f) administering a therapeutic compound comprising said thawed cells to a patient in need thereof.

According to some embodiments, the presently disclosed method further includes the step of (d) charging a fee.

According to some embodiments, the population of cells is not further purified.

It is now disclosed for the first time a cell-based therapeutic agent comprising (a) the population of cells of obtained using any of the presently-disclosed methods, and (b) a pharmaceutically acceptable carrier.

It is now disclosed for the first time a population of cells comprising stem cells and progenitor cells isolated from the umbilical cord tissues by a method that consists essentially of mechanical and enzymatic extraction without prior removal of any blood vessel and/or without prior removal of any vein.

It is now disclosed for the first time a method for preserving a population of cells comprising (a) isolating a population of cells comprising stem cells and progenitor cells from an umbilical cord tissue (i.e one or more umbilical cords) substantially without further purification; and (b) cryopreserving the cells.

Any of the presently disclosed population of cells (for example, derived using a presently-disclosed method) may be administered (for example, in a pharmaceutical composition) to a patient in need thereof, for the treatment of disease (for example, disease that is treatable by tissue regeneration and/or protein replacement and/or coagulation factors).

According to some embodiments, the disease is associated with biological processes selected from the group of processes consisting of cardiac ischemia, osteoporosis, chronic wounds, diabetes, neural degenerative diseases, neural injuries, bone or cartilage injuries, ablated bone marrow, anemia, liver diseases, hair growth, teeth growth, retinal disease or injuries, eye diseases or injuries, ear injuries or diseases muscle degeneration or injury.

According to some embodiments, the administration includes intravenous injection of cells of said population of cells (for example, into specific organs).

According to some embodiments, the patient is in need of a cosmetic therapy selected from the group of cosmetic therapies consisting of filling of skin wrinkles, supporting organs, supporting surgical procedures, treating burns, and treating wounds.

According to some embodiments, the combination between the donor and recipient of the cells of said derived cell population is autologous.

According to some embodiments, the combination between the donor and recipient of the cells of said derived cell population is allogeneic.

It is now disclosed for the first time a population of cells comprising cells having the surface markers SH-2, cells having the surface marker CD31, and cells having the surface markers CD45.

It is now disclosed for the first time a population of cells comprising cells having the surface marker SH-2, and cells having the surface markers CD31.

It is now disclosed for the first time a population of cells comprising cells having the surface marker CD90, and cells having the surface marker VEGFR-2.

It is now disclosed for the first time a population of cells comprising cells having the surface marker SH-2 and cells having the surface marker VEGFR.

It is now disclosed for the first time a population of cells comprising cells having the surface marker CD90 and cells having the surface marker CD31

It is now disclosed for the first time a population of cells comprising cells having the surface marker CD44, cells having the surface marker CD13, cells having the surface marker CD90, cells having the surface marker CD105, cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s.

According to some embodiments, the cells are not activated ex vivo.

According to some embodiments, the cell population comprises hematopoietic cells.

According to some embodiments, the cell population comprises cells having hematopoietic committed lineages.

According to some embodiments, the cell population comprises mesenchymal stem cells. It is now disclosed for the first time a population of cells comprising: (a) a first plurality of stem cells comprising at least one of mesenchymal stem cells and hematapoeitic stem cells from an umbilical cord source; and (b) a second plurality of cells comprising endothelial progenitor cells from walls of a blood vessel of said umbilical cord source.

According to some embodiments, the first plurality includes both said mesenchymal stem cells and said hematapoeitic stem cells.

According to some embodiments, the hematapoeitic stem cells are derived from cord blood.

According to some embodiments, the at least some of said first and second plurality of cells are derived from the same individual.

According to some embodiments, the cells of the presently disclosed populations are human cells.

According to some embodiments, within the population of cells, said mesenchymal stem cells comprise a fraction of the population that is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species as the population of cells, for example, naturally occurring in humans) fraction of mesenchymal stem cells in umbilical cords. In different embodiments, this ‘natural occurring fraction’ may be measured for the case where the umbilical cord contains substantially all umbilical cord blood or substantially no umbilical cord blood

According to some embodiments, within the population of cells, said hematapoeitic stem cells comprise a fraction of the population that is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species in the as the population of cells, for example, naturally occurring in humans) fraction of mesenchymal stem cells in umbilical cords. In different embodiments, this ‘natural occurring fraction’ may be measured for the case where the umbilical cord contains substantially all umbilical cord blood or substantially no umbilical cord blood

According to some embodiments, within the population of cells, said endothelial progenitor cells comprise a fraction of the population that is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species in the as the population of cells, for example, naturally occurring in humans) fraction of endothelial progenitor cells in umbilical cords. In different embodiments, this ‘natural occurring fraction’ may be measured for the case where the umbilical cord contains substantially all umbilical cord blood or substantially no umbilical cord blood

According to some embodiments, a ratio between a number of said mesenchymal stem cells and said hematapoeitic stem cells within the population is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species in the as the population of cells, for example, naturally occurring in humans) ratio within the umbilical cords. In different embodiments, this ‘natural occurring ratio’ may be measured for the case where the umbilical cord contains substantially all umbilical cord blood or substantially no umbilical cord blood

According to some embodiments, a ratio between a number of said mesenchymal stem cells and said epithelial progenitor cells within the population is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species in. the as the population of cells, for example, naturally occurring in humans) ratio within the umbilical cords In different embodiments, this ‘natural occurring ratio’ may be measured for the case where the umbilical cord contains substantially all umbilical cord blood or substantially no umbilical cord blood

According to some embodiments, a ratio between a number of said hematapoeitic stem cells and said epithelial progenitor cells within the population is substantially equal (for example, within a tolerance of 1%, or within a tolerance of 5%, within a tolerance of 10%, or within a tolerance of 30%, or within a tolerance of 50%) to the naturally occurring (for example, naturally occurring in the same species in the as the population of cells, for example, naturally occurring in humans) ratio within the umbilical cords. In different embodiments, this ‘natural occurring ratio’ may be measured for the case where the umbilical cord contains substantially all umbilical cord blood or substantially no umbilical cord blood

It is now disclosed for the first time the presently-disclosed population of cells derived using the presently-disclosed method.

It is now disclosed for the time a therapeutic agent comprising (a) the presently-disclosed population of cells, and b) a pharmaceutically acceptable carrier (for example, for delivering the cells to a specific location).

Embodiments of the present application are also directed to the business process of extracting stem and progenitor cell populations from umbilical cord tissues and their private storage for individuals' future medical needs as well as for clinical use by other individuals. Such cell populations, which are not purified (or minimally purified), will be more effective and more practical candidates in future clinical applications.

Another aspect of the invention is the development of a bank of stem cells that can be tissue typed and banked and expanded as needed.

Another aspect of the invention is the development of cell populations that can be rendered mitotically inactive and then used as feeder cells for establishing and maintaining ES and EG cells from various species.

A further aspect of the invention relates to cell culture technology using the stem cells of the invention in a non-mitotic form as a feeder cell in combination with other stem cells, e.g., embryonic stem cells, capable of growth, transformation and use in treating human or animal disease or in agricultural applications.

A further aspect of the invention relates to cell culture technology using the stem cells of the invention in a treatment for diseases including but not limited to cardiac ischemica, myelomonoblastic leukemia, Parkinson's Disease, stroke, diabetes, and pathologies associated with the connective tissues of the human body.

### Utilization of Stem Cells Provided in the Presently Disclosed Compositions and/or Derived Using a Presently Disclosed Method

It is now disclosed that there are a plethora of applications (in all amniotic animals) for the presently disclosed cell populations, including mesenchymal stem cells, endothelial progenitor cells, and optionally hemapoetic stem cells.

As is disclosed in US 2004/0136967 of Weiss with reference to umbilical cord matrix stem cells derived from Wharton's jelly (paragraphs 0036-0048), it is now disclosed that the presently disclosed populations of cells including a mixture of mesenchymal stem cells and other cells may be used in a variety of applications, including but not limited to:

1) Regenerating tissues which have been damaged through acquired or genetic disease;

2) Treating a patient with damaged tissue or organs with the mixed populations of stem cells combined with a biocompatible carrier suitable for delivering the stem and/or progenitor cells to the damaged tissue sites for correcting, repairing or modifying connective tissue disorders such as the regeneration of damaged muscle;

3) Producing various tissues derived from the mixed populations of cells;

4) Applying the mixed populations of stem cells to an area of connective tissue damage under conditions suitable for differentiating the cells into the type of connective tissue necessary for repair;

5) Various methods or devices for utilizing the mixed populations of stem cells cells in order to enhance hematopoietic cell production; and

6) Methods for using composite grafts of umbilical cord-derived stem cells during bone marrow transplantation.

7) Methods of treating stroke, neurodegenerative diseases, diabetes, vascular conditions.

In some embodiments, implants including the presently-disclosed populations of cells are provided. In some embodiments, kits for carrying out one of the presently-disclosed methods are provided.
Another aspect of the invention is the development of cell populations that can be rendered mitotically inactive and then used as feeder cells for establishing and maintaining ES and EG cells from various species.

### DETAILED DESCRIPTION OF THE INVENTION

The present invention will now be described in terms of specific, example embodiments. It is to be understood that the invention is not limited to the example embodiments disclosed. It should also be understood that not every feature of the compositions, mixtures, populations of cells and methods of producing the same described is necessary to implement the invention as claimed in any particular one of the appended claims. Various elements and features of devices are described to fully enable the invention. It should also be understood that throughout this disclosure, where a process or method is shown or described, the steps of the method may be performed in any order or simultaneously, unless it is clear from the context that one step depends on another being performed first.

### Overview

The present inventor is now disclosing for the first time that, surprisingly, one may obtain useful and novel cell populations and compositions including Wharton's jelly-derived mesenchymal stem cells from umbilical cords without prior removal of any blood vessel and/or without prior removal of any vein Furthermore, the present inventor is now disclosing for the first time, novel compositions of stem cells derived from the umbilical cord

Embodiments of the present invention are directed to populations of stem cells and progenitor cells and their use as therapeutic agents. The cell populations may include populations of cells isolated from the various tissues of the umbilical cord and can include adult stem cells and progenitor cells. Not wishing to be bound by theory, it is noted that the isolation methods, while being sufficient to remove the desired cell populations from the tissues, will, in some embodiments, not include further purification beyond separate collection of umbilical cord blood and remaining cord tissues.

The products of the presently disclosed methods, and the presently disclosed compositions, are useful in a number of applications, including but not limited to regenerative medicine, for screening compounds, for research, and for gene therapy.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Cell Biology: A Laboratory Handbook” Volumes I-III Cellis, J. E., ed. (1994); “Current Protocols in Immunology” Volumes I-III Coligan J. E., ed. (1994); “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods and Enzymology” Vol. 1-317 Academic Press; all of which are incorporated by reference as if fully set forth herein. Other general references are provided throughout this document. The procedures therein are believed to be well known in the art and are provided for the convenience of the reader. All the information contained therein is incorporated herein by reference.

According to some embodiments, the processing of the cord tissue does not include prior removal of the blood vessels or any other tissue, but rather processed as it is, for the collection of all possible stem and progenitor cells. Not wishing to be bound by bound by theory, it is noted that mixtures providing different types of stem and/or progenitor cells may provide a biological synergy.

### DEFINITIONS

Various terms used throughout the specification and claims are defined as set forth below.

Stem cells are undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts.

Stem cells are classified according to their developmental potential as: (1) totipotent; (2) pluripotent; (3) multipotent; (4) oligopotent, and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipolent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

A mesenchymal, placental, cord blood, or other stem cell may be characterized by its cell markers. A variety of cell markers are known. See e.g., Stem Cells: Scientific Progress and Future Research Directions. Department of Health and Human Services. June 2001. http://www.nih.gov/news/stemcell/scireport.htm. Cell markers may be detected by methods known in the art, such as by immunochemistry or flow cytometry. Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics. Antibodies specific to a cell marker may be labeled with a fluorochrome so that it may be detected by the flow cytometer. See, eg., Bonner et al., Rev. Sci. Instrum 43: 404-409, 1972; Herzenberg et al., Immunol. Today 21: 383-390, 2000; Julius et al., PNAS 69: 1934-1938, 1972; Ormerod (ed.), Flow Cytometry: A Practical Approach, Oxford Univ. Press, 1997; Jaroszesli et al, (eds.), Flow Cytometry Protocols in Methods in Molecular Biology No. 91, Humana Press, 1997; Practical Flow Cytometry, 3^{rd} ed., Wiley-Liss, 1995.

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

The stem cells derived from the methods disclosed herein and provided in the compositions described herein may also be cryopreserved. Methods for cyropreserving cells are well known in the art, and any acceptable method is within the scope of the present invention. For example, the cells may be cryopreserved in a solution comprising, for example, dimethyl sulfoxide at a final concentration not exceeding 10%. The cells may also be cryopreserved in a solution comprising dimethyl sulfoxide and/or dextran. Other methods of cryopreserving cells are known in the art.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

As used herein, the phrase differentiates into a mesodermal, ectodermal or endodermal lineage refers to a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to, pleurigenic cells, hepatogenic cells, cells that give rise to the lining of the intestine, and cells that give rise to pancreogenic and splanchogenic cells.

It is noted the mixed populations of stem cells now disclosed may be used in the treatment of any kind of injury due to trauma where tissues need to be replaced or regenerated. Examples of such trauma-related conditions include central nervous system (CNS) injuries, including injuries to the brain, spinal cord, or tissue surrounding the CNS injuries to the peripheral nervous system (PNS), or injuries to any other part of the body. Such trauma may be caused by accident, or may be a normal or abnormal outcome of a medical procedure such as surgery or angioplasty. The trauma may be related to a rupture or occlusion of a blood vessel, for example, in stroke or phlebitis. In specific embodiments, the cells may be used in autologous or allogeneic tissue replacement or regeneration therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues. Injuries may be due to specific conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, ADS dementia, memory loss, amyotrophic lateral sclerosis (ALS), ischemic renal disease, brain or spinal cord trauma, heart-lung bypass, glaucoma, retinal ischemia, retinal trauma, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharide nosis, chronic granulomatous disease and tyrosinemia, Tay-Sachs disease, cancer, tumors or other pathological or neoplastic conditions.

Certain embodiments of the present invention refer to “populations of cells” and methods for producing the same. As used herein, a “population of cells” is defined to exclude a mass of tissue (for example, extra-cellular matrix tissue, blood vessels or any other tissue). Exemplary populations of cells include isolated populations of cells, populations of cells (i.e. individual cells) suspended in solution, and populations of cells that are suspended in solution and then cryopresrved.

The term “Umbilical Cord Matrix Stem Cell” as used herein refers to either:

1) A pluripotent, or lineage-uncommitted progenitor cell, typically referred to in the art as a “stem cell” derived from the umbilical cord matrix, other than a cord blood cell source. Such a cell is potentially capable of an unlimited number of mitotic divisions to either renew its line or to produce progeny cells which will differentiate into the mature functional cells that will constitute most of the tissues of an organism such as tissues derived from any of the three germ layers (ectoderm, endoderm, neuroderm) and germ cells; or

2) A lineage-committed progeny cell produced from the mitotic division of a stem cell of the invention that can eventually differentiate into any of the three germ layer derivatives or germ cells. Unlike the stem cell from which it is derived, the lineage-committed progeny cell is generally considered to be incapable of an unlimited number of mitotic divisions to produce other progeny cells.

Embodiments of the invention are directed primarily to compositions and methods for the production of mixtures of stem cells and their derivatives such as any of the three germ layer derivatives or germ cell lines and cells, tissues and organs. However the invention may also be practiced so as to produce stem cells and their derivatives in any amniote in need thereof.

According to the invention, stem cells may be obtained from an umbilical cord collected from a subject's own umbilical cord. Alternatively, it may be advantageous to obtain stem cells from an umbilical cord obtained from an umbilical cord associated with a species specific or species related developing fetus or newborn, where the subject in need of treatment is one of the parents of the fetus or newborn. Another scenario involves banking and tissue typing and cataloging so that any individual in need of a stem cell graft might find an appropriate match.

Alternatively, because of the primitive nature of cells isolated from the umbilical cord, immune rejection of the cells of the invention or the new tissue produced therefrom may be minimized. As a result, such cells may be useful as “ubiquitous donor cells” for the production of new cells and tissue for use in any subject in need thereof.

The term “Wharton's Jelly,” also known as inter-laminar jelly, as used herein, is a subset of the umbilical cord matrix, and refers to a mucous-connective tissue substance found in the umbilical cord. The components of Wharton's Jelly include a mucous connective tissue in which are found myofibroblasts, fibroblasts, collagen fibers and an amorphous ground substance composed of hyaluronic acid and possibly other as yet uncharacterized cell populations. Wharton's Jelly is one component of the umbilical cord.

The term “Umbilical Cord” as used herein, refers to the Umbilical cord-structure enclosing the body stalk, and the stalks of the yolk sac and allantois. The enclosing membrane of the umbilical cord is formed by the folding of the amnion.

For the purpose of this disclosure, the term “feeder cell” or “feeder cell culture”, as used herein, refers to cells that provide a co-stimulating function in conjunction with typically the other stem cell cultures, not necessarily the cells of this invention. A feeder cell can be obtained by culture techniques known in the art such as that shown by Weaver et al., Blood 82:1981-1984, 1993. Feeder cell cultures can be stored by cryopreservation in liquid nitrogen until use. Prior to the use of such feeder cells, for the purpose of maintaining a culture of stem cells (other than the feeder cells), such feeder cells are stabilized to promote the isolation and maintenance of stem cell cultures. “Homing potential” refers to an inherent capacity of a cell to be targeted to specific locations for therapeutic function or purpose.

As used herein the term “ex-vivo” refers to cells removed from a living organism and are propagated outside the organism (e.g., in a test tube). As used herein, the term “ex-vivo”, however, does not refer to cells known to propagate only in-vitro, such as various cell lines (e.g., HL-60, MEL, HeLa, etc.).

As used herein the term “inhibiting” refers to slowing, decreasing, delaying, preventing or abolishing.

As used herein the term “differentiation” refers to a change from relatively generalized to specialized kinds during development Cell differentiation of various cell lineages is a well-documented process and requires no further description herein. As used herein the term “differentiation” is distinct from maturation which is a process, although some times associated with cell division, in which a specific cell type mature to function and then dies, e.g., via programmed cell death (apoptosis).

As used herein the phrase “cell expansion” refers to a process of cell proliferation substantially devoid of cell differentiation. Cells that undergo expansion hence maintain their renewal properties and are oftentimes referred to herein as renewable cells, e.g., renewable stem cells.

### Exemplary Protocol

In order to isolate the stem cells according to the invention, umbilical cord is obtained under sterile conditions immediately following the termination of pregnancy (either full term or pre-term). The umbilical cord or a section thereof, according to one embodiment of the invention, may be transported from the site of the delivery to a laboratory in a sterile container containing a preservative medium. One example of such a preservative medium is Dulbecco's Modified Eagle's Medium (DEM) with HEPES buffer.

The umbilical cord is preferably maintained and handled under sterile conditions prior to and during the collection of the cell population including stem cells from the may additionally be surface-sterilized by brief surface treatment of the cord with, for example, an aqueous (70% ethanol) solution or betadine, followed by a rinse with sterile, distilled water. The umbilical cord can be briefly stored for up to about three hours at about 3-5.degree. C., but not frozen, prior to extraction of cells including stem cells and/or progenitor cells from the umbilical cord.

Thus, an umbilical cord (the entire cord or a section thereof) is obtained either before or after removal of the cord blood, The cord's two ends are ligtated, and the cord is immersed in a buffered medium. The chord is then transferred to the processing lab and processed within 48 hours. No other procedure such as removal of blood vessels is performed and the entire cord is further processed. The cord is then washed in saline or similar fluid and mechanically chopped into small pieces, or up to the formation of a paste-like material. The material is then transferred into a magnetic stirrer container and incubated for 2-4 hours in collagenase and hyaluronidase solution. The cell suspension is then centrifuged and the cells are suspended in either culture media or in culture media containing freezing reagent. Culture medium used with the cells can include serum or plasma as required. In an embodiment, storage can include the use of low temperatures in a cryopreservation method.

### Stem Cell Bank

The invention includes a method of generating a bank of stem cells by obtaining matrix cells from the umbilical cord, fractionating the matrix into a fraction enriched with a stem cell and culturing the stem cells in a culture medium containing one or more growth factors. By this process, the stem cells will undergo mitotic expansion. Alternatively, a bank of the umbilical cord itself and/or unfractionated cells may be maintained for later obtaining matrix cells.

The invention contemplates the establishment and maintenance of cultures of stem cells as well as mixed cultures comprising stem cells, mature cells and mature cell lines. Once a culture of stem cells or a mixed culture of stem cells and mature cells is established, the cultures should be transferred to fresh medium when sufficient cell density is reached. By this means, formation of a monolayer of cells should be prevented or minimized, for example, by transferring a portion of the cells to a new culture vessel and into fresh medium. Alternatively, the culture system can be agitated prevent the cells from sticking or grown in Teflon-coated culture bags.

Once the cells of the invention have been established in culture, as described above, they may be maintained or stored in “cell banks” comprising either continuous in vitro cultures of cells requiring regular transfer, or, preferably, cells which have been cryopreserved. Cryopreservation of cells of the invention may be carried out according to known methods, such as those described in Doyle et al., 1995, Cell and Tissue Culture. For example, but not by way of limitation, cells may be suspended in a “freeze medium” such as, for example, culture medium further comprising 15-20% FBS and 10% dimethylsulfoxide (DMSO), with or without 5-10% glycerol, at a density, for example, of about 4-10.times.10.sup.6 cells-ml.sup.−1. The cells are dispensed into glass or plastic ampoules (Nunc) that are then sealed and transferred to the freezing chamber of a programmable freezer. The optimal rate of freezing may be determined empirically. For example, a freezing program that gives a change in temperature of about −1.degree. C.-min.sup.−1 through the heat of fusion may be used. Once the ampoules have reached about −180.degree. C., they are transferred to a liquid nitrogen storage area. Cryopreserved cells can be stored for a period of years, though they should be checked at least every 5 years for maintenance of viability.

The cryopreserved cells of the invention constitute a bank of cells, portions of which can be “withdrawn” by thawing and then used to produce new stem cells, etc. as needed. Thawing should generally be carried out rapidly, for example, by transferring an ampoule from liquid nitrogen to a 37 degree C. water bath. The thawed contents of the ampoule should be immediately transferred under sterile conditions to a culture vessel containing an appropriate medium such as RPMI 1640, DMEM conditioned with 20% FBS. The cells in the culture medium are preferably adjusted to an initial density of about 3.times.10.sup.5 cells-ml.sup.−1-6.times.10.sup.5 cells-ml.sup.−1 so that the cells can condition the medium as soon as possible, thereby preventing a protracted lag phase. Once in culture, the cells may be examined daily, for example, with an inverted microscope to detect cell proliferation, and sub-cultured as soon as they reach an appropriate density.

The cells of the invention may be withdrawn from the bank as needed, and used for the production of new tissue either in vitro, or in vivo, for example, by direct administration of cells to the site where new tissue is needed. As described supra, the cells of the invention may be used to produce new tissue for use in a subject where the cells were originally isolated from that subject's umbilical cord (autologous).

Alternatively, the cells of the invention may be used as ubiquitous donor cells, i.e., to produce new tissue for use in any subject heterologous).

### Feeder Culture Cells

In an embodiment, the stem cells of the invention can be employed to create feeder cell culture materials. The present cells can be used for species specific or other appropriate feeder culture cells for ES, EG or other stem cells (for example, neural stem cells).

The stem cells of the application can be used in the form of the feeder cells that remain alive, that can produce growth factor and other materials for maintaining culture materials, but that do not divide or grow. The feeder cells can be prevented from beginning or conducting a mitotic process by using irradiation, chemical treatment or another technique that can prevent such processes. After performing such a technique, the feeder cells are alive and can function, but will not divide or grow. In using feeder cells to culture the stem cells of the invention, the feeder cells can, for example, provide growth factors to the growing totipotent, pluripotent, or multipotent stem cells. Growth factors can be added to the culture if the feeder cells are incapable of providing sufficient quantities. The feeder cells can be grown and selected such that they express selected growth factors, for example, factors useful in the manufacture of neural, epithelial or other such desirable cell types and characteristics.

In an embodiment, the feeder cells are treated to prevent mitotic transformations or are inactivated prior to use. In an embodiment, the feeder cells are inactivated using radiation or chemical treatment. Radiation useful for such transformation can include X-radiation, gamma radiation, or electron radiation from appropriate sources. X-radiation can be used from electronic generation or from agents such as cobalt or cesium. Chemical treatments can be made with agents such as Mitomycin C. The resulting inactivated feeder cells can be cultured in culturing PGC's, for example, for 24 hours prior to culturing with a stem cell material. Fresh isolates can be taken on a regular basis to ensure that the cells are continually available.

Feeder cell layers can be useful for both the isolation of stem cell lines from embryos and other sources and for the routine maintenance of established cell lines. Mixtures of different types of umbilical cord-derived cells can be typically plated to give a uniform monolayer of cells onto which the stem cells are seeded. Species-specific feeder cells can provide adequate growth conditions for successful culture development.

The stem cells can be isolated for feeder cell purposes, and other purposes. Once isolated from the umbilical cord, the mixtures of different types of stem and/or progenitor cells can be dispersed and suspended in an aqueous medium such as trypsin EDTA solution. Adding DMEM solution plus serum can neutralize the trypsin. The contents of the dish are transferred to a 10 ml conical tube. The tube is then centrifuged or held stationary to settle large particulate materials. Different types of umbilical-cord derived stem cells in the supernatant can be plated with standard growth medium and maintained with conventional culture technique.

The use of the stem cells of this invention as a feeder cell in stem cell cultures provides a number of advantages. First, the cells are stem cells and provide growth factors that are applicable to other human stem cells from other sources such as embryonic sources, adult sources such as blood sources, adipose or fat sources and other human sources. Further, the mixture of different types of umbilical cord-derived stem cells provides a final cell culture in which the feeder cells do not prevent the use of the cultured stem cells from application in human use. Such feeder cell cultures can be made using known techniques.

### Uses of the Mixtures of Stern Cell and/or Progenitor Cells Derived From the Umbilical Cord

The cells of the invention may be used in human or animal medicine, agriculturally important species and in research. For example the cells of the invention may be used to treat subjects requiring the repair or replacement of body tissues resulting from disease or trauma. Treatment may entail the use of the cells of the invention to produce new tissue, and the use of the tissue thus produced, according to any method presently known in the art or to be developed in the future. For example, the cells of the invention may be implanted, injected or otherwise administered directly to the site of tissue damage so that they will produce new tissue in vivo.

The culturing and transplant of stem cells and/or progenitor cells into mammals is well known in the art. Thus, protocols for in vitro cell differentiation and/or expansion of stem cells and/or progenitor cells, protocols for tissue engineering, and protocols for transplant of stem cells and/or progenitor cells into mammals such as rodents have been published. For example, one or more examples published in US 2004/0136967 of Weiss, US 2005/0148074 of Davies, US 2004/0258670 of Laughlin, among others It is now disclosed that one or more of these published protocols (for example, published in “Example Sections” or any other section of these published patent applications) may be suitably modified for use with one or more of the presently-disclosed mixtures and populations of cells.

In general, it is noted that the stem cells derived from the umbilical cord, the mature cells produced from these stem cells, the cell lines derived from these stem cells, and the tissue of the invention can be used:
(1) to screen for the efficacy and/or cytotoxicity of compounds, allergens, growth/regulatory factors, pharmaceutical compounds, etc.;
(2) to elucidate the mechanism of certain diseases;
(3) to study the mechanism by which drugs and/or growth factors operate;
(4) to diagnose, monitor and treat cancer in a patient;
(5) for gene therapy;
(6) to produce biologically active products;
(7) to target delivery of a drug to a specific tissue. To do this they may first be engineered to produce the drug;
(8) to be utilized for their homing ability that permits the cells to migrate from a treatment location to a specific target location (for example, where a pathology or abnormal condition exists);
(9) to produce beta cells for insulin production; and
(10) for transplantation to treat neurodegenerative disease, stroke, reperfusion injuries, and other vascular conditions.
(11) to produce transgenic animals by the method of injecting transgenic mixtures of cells including umbilical cord-derived stem cells into early embryos (morulae and/or blastocysts) to produce chimeric embryos and individuals
(12) to preserve or rescue the genetic material of endangered species or genetic stocks of strains of agricultural or laboratory animals.

### (1) Screening Effectiveness and Cytotoxicity of Compounds

The cells and tissues of the invention may be used in vitro to screen a wide variety of compounds for effectiveness and cytotoxicity of pharmaceutical agents, growth/regulatory factors, anti-inflammatory agents, etc. To this end, the cells of the invention, or tissue cultures described above, are maintained in vitro and exposed to the compound to be tested. The activity of a cytotoxic compound can be measured by its ability to damage or kill cells in culture. This may readily be assessed by vital staining techniques. Analyzing the number of living cells in vitro, e.g., by total cell counts, may assess the effect of growth/regulatory factors and differential cell counts. This may be accomplished using standard cytological and/or histological techniques, including the use of immunocytochemical techniques employing antibodies that define type-specific cellular antigens. The effect of various drugs on the cells of the invention either in suspension culture or in the three-dimensional system described above may be assessed.

### (2) Elucidate the Mechanism of Certain Diseases

The cells and tissues of the invention may be used as model systems for the study of physiological or pathological conditions. For example, the cells and tissues of the invention may be used to determine the nutritional requirements of a tissue under different physical conditions, e.g., intermittent pressurization, and by pumping action of nutrient medium into and out of the tissue construct. This may be especially useful in studying underlying causes for age-related or injury-related disorders.

(3) Study the Mechanism by which Drugs and/or Growth Factors Operate

The stem cells, cell lines, mature cells and tissues of the invention may also be used to study the mechanism of action of morphagens, chemokines, cytokines, and other pro-inflammatory mediators, e.g., IL-1, TNF and prostaglandins. In addition, cytotoxic and/or pharmaceutical agents can be screened for those that are most efficacious for a particular application. Agents which prove to be efficacious in vitro could then be used to treat the patient therapeutically.

### (4) Diagnosis, Monitoring and Treatment of Cancer or Cancer Cells, Tissues or Symptoms

Based upon their tropism for tissue pathology, the cells and tissues of the invention may be used to diagnose, treat or monitor cancer or reduce its symptoms.

### (5) Gene Therapy

The cells and tissues of the present invention may afford a vehicle for introducing genes and gene products in vivo to assist or improve the results of implantation and/or for use in gene therapies. The following description is directed to the genetic engineering of any of the cells of the invention or tissues produced therefrom.

Cells which express a gene product of interest, or the tissue produced in vitro therefrom, can be implanted into a subject who is otherwise deficient in that gene product. For example, genes that express a product capable of preventing or ameliorating symptoms of various types of diseases, such as those involved in preventing inflammatory reactions, may be under-expressed or down-regulated under disease conditions. Alternatively, the activity of gene products may be diminished, leading to the manifestation of some or all of the pathological conditions associated with a disease. In either case, the level of active gene product can be increased by gene therapy, i.e., by genetically engineering cells of the invention to produce active gene product and implanting the engineered cells, or tissues made therefrom, into a subject in need thereof. A related application foreseen in agricultural or other animals is the delivery of a product that enhances growth, maturation, reproduction, etc. The products of interest may be delivered over the long term or alternatively and transiently to achieve the desired effect

Alternatively, the cells of the invention can be genetically engineered to produce a gene product that would serve to stimulate tissue or organ production such as, for example, BMP-13 or TGF-.beta. Also, for example, the cells of the invention may be engineered to express the gene encoding the human complement regulatory protein that prevents rejection of a graft by the host. See, for example, McCurry et al., 1995, Nature Medicine 1:423-427.

A related application foreseen in animals is the use of these cells to generate transgenic animals using methods that have been developed for mouse ES cells. The chimeric animals will be used to establish transgenic animal lines. Another related application foreseen in animals is the use of these cells to generate chimeric animals that produce useful compounds.

Methods that may be useful to genetically engineer the cells of the invention are well-known in the art. For example, a recombinant DNA construct or vector containing the gene of interest may be constructed and used to transform or transfect one or more cells of the invention. Such transformed or transfected cells that carry the gene of interest, and that are capable of expressing said gene, are selected and clonally expanded in culture. Methods for preparing DNA constructs containing the gene of interest, for transforming or transfecting cells, and for selecting cells carrying and expressing the gene of interest are well-known in the art. See, for example, the techniques described in Maniatis et al., 1989, Molecular Cloning, A Laboratory Manual, Cold Spring Harbor Laboratory Press, Cold Spring Harbor, N.Y.; Ausubel et al., 1989, Current Protocols in Molecular Biology, Greene Publishing Associates & Wiley Interscience, N.Y.; and Sambrook et al., 1989, Molecular Cloning: A Laboratory Manual, 2nd Ed., Cold Spring Harbor Laboratory Press, Cold Spring Harbor, N.Y. In addition, the transkaryotic implantation technique described by Seldon et al., 1987, Science 236:714-718, may be useful. All of these publications are incorporated herein by reference.

The cells of the invention can be engineered using any of a variety of vectors including, but not limited to, integrating viral vectors, e.g., retrovirus vector or adeno-associated viral vectors, or non-integrating replicating vectors, e.g., papilloma virus vectors, SV40 vectors, adenoviral vectors; or replication-defective viral vectors. Other methods of introducing DNA into cells include the use of liposomes, electroporation, a particle gun, or by direct DNA injection.

Host cells are preferably transformed or transfected with DNA controlled by, i.e., in operative association with, one or more appropriate expression control elements such as promoter or enhancer sequences, transcription terminators, polyadenylation sites, among others, and a selectable marker. Following the introduction of the foreign DNA, engineered cells may be allowed to grow in enriched media and then switched to selective media. The selectable marker in the foreign DNA confers resistance to the selection and allows cells to stably integrate the foreign DNA as, for example, on a plasmid, into their chromosomes and grow to form foci which, in turn, can be cloned and expanded into cell lines. This method can be advantageously used to engineer cell lines that express the gene product.

Any promoter may be used to drive the expression of the inserted gene. For example, viral promoters include but are not limited to the CMV promoter/enhancer, SV 40, papillomavirus, Epstein-Barr virus, elastin gene promoter and .beta-globin. Preferably, the control elements used to control expression of the gene of interest should allow for the regulated expression of the gene so that the product is synthesized only when needed in vivo. If transient expression is desired, constitutive promoters are preferably used in a non-integrating and/or replication-defective vector. Alternatively, inducible promoters could be used to drive the expression of the inserted gene when necessary. Inducible promoters include, but are not limited to, those associated with metallothionein and heat shock protein.

Examples of transcriptional control regions that exhibit tissue specificity which have been described and could be used include but are not limited to: elastase I gene control region, which is active in pancreatic acinar cells (Swit et al., 1984, Cell 38:639-646; Ornitz et al., 1986, Cold Spring Harbor Symp. Quant. Biol. 50:399-409; MacDonald, 1987, Hepatology 7:425-515); insulin gene control region, which is active in pancreatic beta cells (Hanahan, 1985, Nature 315:115-122); immunoglobulin gene control region, which is active in lymphoid cells (Grosschedl et al., 1984, Cell 3S:647-658; Adams et al., 1985, Nature 318:533-538; Alexander et al., 1987, Mol. Cell. Biol. 7:1436-1444); myelin basic protein gene control region, which is active in oligodendrocyte cells in the brain (Readhead et al., 1987, Cell 48:703-712); myosin light chain-2 gene control region, which is active in skeletal muscle (Shani, 1985, Nature 314:283-286); and gonadotropic releasing hormone gene control region, which is active in the hypothalamus (Mason et al., 1986, Science 234:1372-1378).

The cells of the invention may be genetically engineered to “knock out” expression of factors that promote inflammation or rejection at the implant site. Negative modulatory techniques for the reduction of target gene expression levels or target gene product activity levels are discussed below. “Negative modulation,” as used herein, refers to a reduction in the level and/or activity of target gene product relative to the level and/or activity of the target gene product in the absence of the modulatory treatment. The expression of a gene native to a specific cell can be reduced or knocked out using a number of techniques including, for example, inhibition of expression by inactivating the gene completely (commonly termed “knockout”) using the homologous recombination technique. Usually, an exon encoding an important region of the protein (or an exon 5′ to that region) is interrupted by a positive selectable marker, e.g., neo, preventing the production of normal MRNA from the target gene and resulting in inactivation of the gene. A gene may also be inactivated by creating a deletion in part of a gene, or by deleting the entire gene. By using a construct with two regions of homology to the target gene that are far apart in the genome, the sequences intervening the two regions can be deleted (Mombaerts et al., 1991, Proc. Nat Acad. Sci. U.S.A. 88:3084-3087).

Antisense and ribozyme molecules that inhibit expression of the target gene can also be used in accordance with the invention to reduce the level of target gene activity. For example, antisense RNA, small interfering RNA (siRNA), and ribozyme molecules that inhibit the expression of major histocompatibility gene complexes (HLA) have been shown to be most versatile with respect to immune responses. Still further, triple helix molecules can be utilized in reducing the level of target gene activity. These techniques are described in detail by L. G. Davis et al. (eds), 1994, Basic Methods in Molecular Biology, 2nd ed., Appleton & Lange, Norwalk, Conn., which is incorporated herein by reference.

Once the cells of the invention have been genetically engineered, they may be directly implanted into the patient to allow for the amelioration of the symptoms of disease by, for example, producing an anti-inflammatory gene product such as, for example, peptides or polypeptides corresponding to the idiotype of neutralizing antibodies for GM-CSF, TNF, IL-1, IL-2, or other inflammatory cytokines. Alternatively, the genetically engineered cells may be used to produce new tissue in vitro, which is then implanted in the subject, as described supra.

The use of the compositions and methods of the invention in gene therapy has a number of advantages. Firstly, since the culture comprises eukaryotic cells, the gene product will likely be properly expressed and processed to form an active product. Secondly, gene therapy techniques are generally useful where the number of transfected cells can be substantially increased to be of clinical value, relevance, and utility. Thus, for example, the three-dimensional culture described supra allows for mitotic expansion of the number of transfected cells and amplification of the gene product to levels that may be efficacious in treating congenital or acquired disease. Transplant of HLA matched cells, used banked cells, etc. are all advantages.

### (6) Production of Biological Molecules

In a further embodiment, the cells of the invention can be cultured in vitro to produce biological products in high yield. For example, such cells, which either naturally produce a particular biological product of interest (e.g., a growth factor, regulatory factor, or peptide hormone etc.), or have been genetically engineered to produce a biological product, could be clonally expanded using, for example, the three-dimensional culture system described above. If the cells excrete the biological product into the nutrient medium, the product can be readily isolated from the spent or conditioned medium using standard separation techniques, e.g., such as differential protein precipitation, ion-exchange chromatography, gel filtration chromatography, electrophoresis, and HPLC, to name but a few. A “bioreactor” may be used to take advantage of the flow method for feeding, for example, a three-dimensional culture in vitro. Essentially, as fresh media is passed through the three-dimensional culture, the biological product is washed out of the culture and may then be isolated from the outflow, as above.

Alternatively, a biological product of interest may remain within the cell and, thus, its collection may require that the cells be lysed. The biological product may then be purified using any one or more of the above-listed techniques.

### (7) Targeted Drug Delivery

The umbilical-cord derived populations of cells including stem cells and/or progenitor cells of the present invention can be used to target delivery of a drug to a specific tissue. To do this they can first be engineered to produce the drug. A foreign gene is integrated in vitro into the genome of the umbilical cord matrix stem cells by lipofection or electroporation, a foreign protein or peptide is expressed, and the stem. cells are introduced in the host tissue either as undifferentiated cells or after differentiation in vitro. The engineered stem cells can be cellular isografts, allografts or xenografts. In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

All references cited herein are incorporated by reference in their entirety. Citation of a reference does not constitute an admission that the reference is prior art.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

1-61. (canceled)

62) A method of deriving a population of cells, the method comprising:
a) obtaining one or more umbilical cords, each said umbilical cord comprising a respective at least one or more umbilical cord blood vessels and respective umbilical cord matrix; or possibly a number of cords for batch production, or possibly a fraction of a cord;
b) for each said umbilical cord, mechanically disrupting at least a portion of a respective said umbilical cord blood vessel and at least a portion of respective said umbilical cord matrix to produce a mixture including umbilical cord blood vessel matter and umbilical cord matrix matter; and
c) deriving the population of cells including umbilical cord matrix-derived cells and umbilical cord blood vessel-derived cells from said mixture;
d) The method may include chopping said cord or more umbilical cords into a plurality of small pieces; The method includes the possibility of forming a paste-like material from the cord or cords and possible enzymatic extraction, deriving said population from said paste-like material. The method may also include a plurality of said umbilical cords within a vessel and subjected to a batch process including at least one of said disrupting and enzymatic extraction and said deriving within said vessel.

63) The method of claim 62, further comprising cryopreserving at least a portion of said population of cells.

64) The method of claim 63, further comprising the steps of thawing said cryopreserved cells and administering a therapeutic agent comprising said thawed cells to a patient in need thereof.

65) The method of claim 62, further comprising the step of charging a fee.

66) The method of claim 62, further comprising the step of culturing the population of cells, and/or activation with or without purification, or without any culturing or activation ex vivo

67) A population of cells derived by any of the methods of claim 62. The said population of cells may have one or more of combinations of the following surface markers: SH2, CD31, CD45, CD90, VEGFR-2, CD44, CD13, CD105, ABCG2, CD34, CD133, C117, CD135, CXCR4, VE-Cadherin, MAC-1, CD11, CD14, CD56, Tie-2s. The said population may comprise of one or more of the following progenitors: Hematopoietic, mesenchymal, endothelial, pluripotent stem cells.

68) A cell-based therapeutic agent comprising:
a) the population of cells of claim 67; and
b) a pharmaceutically acceptable carrier.

69) A population of cells comprising stem cells and progenitor cells isolated from the umbilical cord tissues by a method that consists essentially of mechanical and enzymatic extraction without prior removal of any blood vessel, and the method for preserving these cell populations.

70) The method of claim 62, further comprising administering a therapeutic agent, either by i.v administration or by intra organ injection, or by other methods, comprising said derived population of cells to a patient in need thereof, such as for treatment of a disease associated with biological processes selected from the group of processes consisting of cardiac ischemia, osteoporosis, chronic wounds, diabetes, neural degenerative diseases, neural injuries, bone or cartilage injuries, ablated bone marrow, graft versus host disease, anemia, liver diseases, hair growth, teeth growth, retinal disease or injuries, eye diseases or injuries, car injuries or diseases muscle degeneration or injury and skin treatments and any disease or condition that is treatable by tissue regeneration.

71) The combination between the donor and recipient of the cells of said derived cell population is either autologous or allogeneic.

72) The method of claim 62, wherein the patient is in need of a cosmetic treatment or therapy selected from the group of cosmetic therapies consisting of filling of skin wrinkles, supporting organs, supporting surgical procedures, treating burns, cosmetic skin treatment, and treating wounds. The combination between the donor and recipient of the cells of said derived cell population is either autologous or allogeneic.

73) The population of cells of claim 67, wherein a ratio between a number of said mesenchymal stem cells and said hematopoeitic stem cells and said endothelial progenitor cells within the population is substantially equal to the naturally occurring ratio within the umbilical cords.

74) Utilizing the population of cells of claim 67, for screening compounds, for research, and for gene therapy.
